(19) United States
(12) Patent Application Publication
(10) Pub. No.: US 2003/0004435 A1
(43) Pub. Date: Jan. 2, 2003

(54) Device for collecting cellular & DNA specimens

(76) Inventors: Paul G. Crawford, Shreveport, LA (US); Jacque T. LaBarre, Shreveport, LA (US); J. S. Heard, Shreveport, LA (US)

Correspondence Address: John M. Harrison, 2139 E. Bert Kouns, Shreveport, LA, 71105, US

(21) Appl. No.: 10/176,927

(22) Filed: Jun. 21, 2002

Related U.S. Application Data
(60) Provisional application No. 60/301,313, filed on Jun. 28, 2001.

(51) Int. Cl. A61B 10/00

(52) U.S. Cl. 600/572

(57) ABSTRACT

A device which is suitable for collecting cellular and DNA specimens from the upper vagina and lower cervix of a female patient's reproductive tract. The device can be self-administered by the patient and typically includes an expandible, cell-collecting insert disposed in a compressed configuration in a housing which is initially inserted in the patient's vagina. Upon controlled release from the housing, the insert expands into a generally spherical shape to contact the upper vaginal and lower cervical walls and remains in position for a selected period of time, such as eight hours, during which time some of the vaginal and cervical cells become trapped on the insert. After removal of the insert from the vagina, the insert is placed in a suitable specimen container and the cells thereon are tested for abnormal cytology or various medical conditions such as cancer or sexually transmitted diseases.

### CROSS-REFERENCE TO RELATED APPLICATIONS

This application claims the benefit of prior filed copending U.S. Provisional Patent Application Serial No. 60/301,313, filed Jun. 28, 2001.

### BACKGROUND OF THE INVENTION

Field of the Invention

This invention relates to cell sampling devices and more particularly, to a new and improved device for collecting cellular specimens from biological tissue surfaces and which is suitably adapted for collecting cellular and DNA specimens from the vagina and cervix of the female reproductive tract. The device can be self-administered by a female gynecological patient and includes a cell-collecting insert which in typical use is discharged from a tubular housing into the vagina and expands into a generally spherical or other geometrical configuration for contacting the upper vaginal and lower cervical walls of the patient's reproductive tract. The insert is maintained in position for a selected period of time to collect cells from the vagina and cervix, after which the insert is removed from the vagina, and the cell samples collected thereon are removed and tested for various medical conditions such as cancer or sexually-transmitted diseases or cytological or DNA aberrations.

The most common method of retrieving upper vaginal and cervical cell and DNA specimens requires a visit to a physician's office for a speculum-guided pelvic exam. The facility for retrieving such specimens in a home setting, on the other hand, would have multiple uses including, but not limited to, cytologic studies and DNA analysis. Such a technique would afford privacy to patients and result in increased compliance with recommendations for periodic medical evaluation.

Various types of devices for retrieving tissue or cell samples are known in the art and typical of these devices are those detailed in the following U.S. patents: U.S. Pat. No. 4,633,886, dated Jan. 6, 1987, to Bucaro, Jr., U.S. Pat. No. 4,945,921, dated Aug. 7, 1990, to Okimoto; U.S. Pat. No. 5,091,316, dated Feb. 25, 1992, to Monthony, et al.; U.S. Pat. No. 5,231,992, dated Aug. 3, 1993, to Leon; U.S. Pat. No. 5,445,164, dated Aug. 29, 1995, to Worthen, et al; U.S. Pat. No. 6,155,990, dated Dec. 5, 2000, to Fournier; and U.S. Pat. No. 6,174,293, dated Jan. 16, 2001, to Buck, et al.

An object of the present invention is to provide a device which is suitable for collecting cellular and DNA specimens from biological tissue surfaces.

Another object of the invention is to provide a device which is suitably adapted for collecting cellular and DNA specimens from the reproductive tract of a gynecological patient.

Still another object of this invention is to provide a device which is capable of collecting cellular and DNA specimens from the upper vagina and lower cervix of a gynecological patient, which device may be self-administered by the patient.

Yet another object of the invention is to provide a device which includes an expandible, cell-trapping insert which may be selectively discharged by a gynecological patient into her vagina and remains in place against the upper vaginal and lower cervical walls of the patient's reproductive tract for a selected period of time to collect vaginal and cervical cells for testing of abnormal cytology, cancer, sexually transmitted diseases and the like.

Another object of this invention is to provide a method of collecting cell samples from cellular surfaces for analysis and is suitably adapted for collecting cell samples from the vagina and cervix of a gynecological patient for cytological and DNA analysis.

### SUMMARY OF THE INVENTION

These and other objects of the invention are provided in a device for collecting cellular and DNA specimens from biological tissue surfaces, which device is particularly suitable for self-administration by a gynecological patient in obtaining cellular and DNA specimens from the upper vagina and lower cervix. The device typically includes a housing having an expandible, cell-collecting insert initially confined in a compressed configuration in the housing and which housing, in typical use of the device, is initially inserted by a gynecological patient into her vagina. Upon patient-controlled discharge of the insert from the housing and into the patient's vagina, the insert expands into a generally spherical or other geometrical configuration to contact the upper vaginal and lower cervical walls and remains in position for a selected period of time, such as eight hours or overnight, during which time some of the vaginal and cervical cells become trapped on the insert. After the patient removes the insert from her vagina, the insert is typically placed in a suitable specimen container and delivered to a testing facility, whereupon the cells and DNA immobilized on the insert are removed therefrom and tested for various medical conditions such as cancer, sexually transmitted diseases or chromosomal or other cytological aberrations.

### BRIEF DESCRIPTION OF THE DRAWINGS

The invention will be better understood by reference to the accompanying drawings, wherein:

FIG. 1 is a perspective view of an illustrative embodiment of a device for collecting cellular and DNA specimens of this invention;

FIG. 2 is an exploded, perspective view of a device for collecting cellular and DNA specimens;

FIG. 3 is a side view, partially in section, of a device for collecting cellular and DNA specimens, more particularly illustrating insertion of the device into the vagina (in section) of a female patient's reproductive tract prior to releasing the insert element of the device into the vagina;

FIG. 4 is a side view, partially in section, of the device illustrated in FIG. 3, illustrating expansion of the insert element of the device as the insert is discharged into the vagina from the housing; and

FIG. 5 is a side view, partially in section, of the device illustrated in FIGS. 3 and 4, illustrating contact of the expanded insert with the upper vaginal and lower cervical walls of the patient's reproductive tract.

### DESCRIPTION OF THE PREFERRED EMBODIMENTS

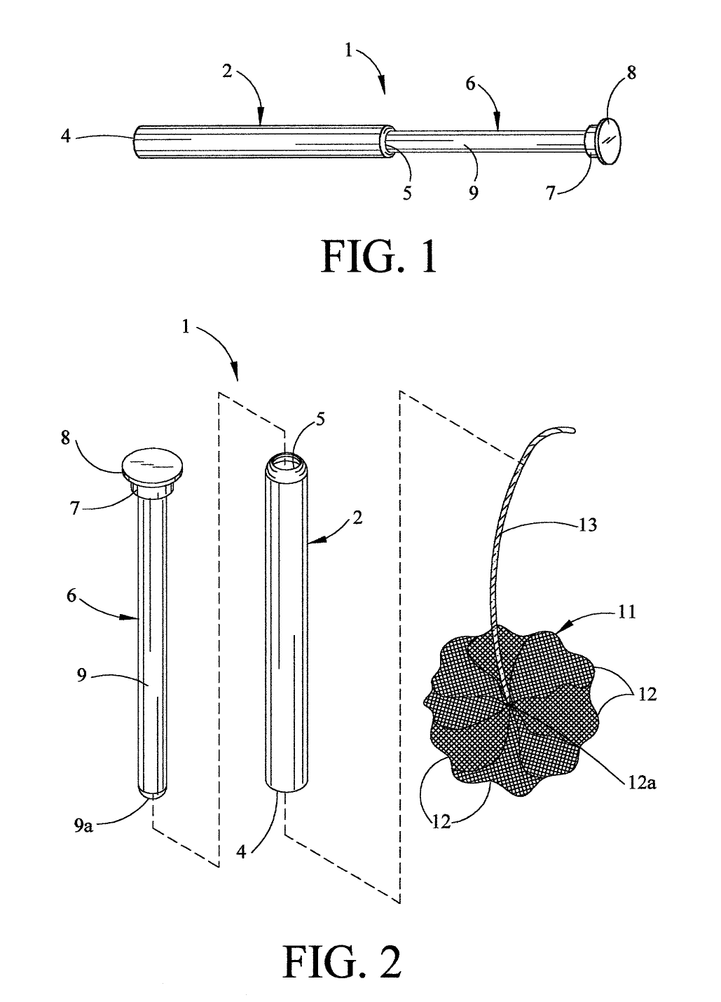

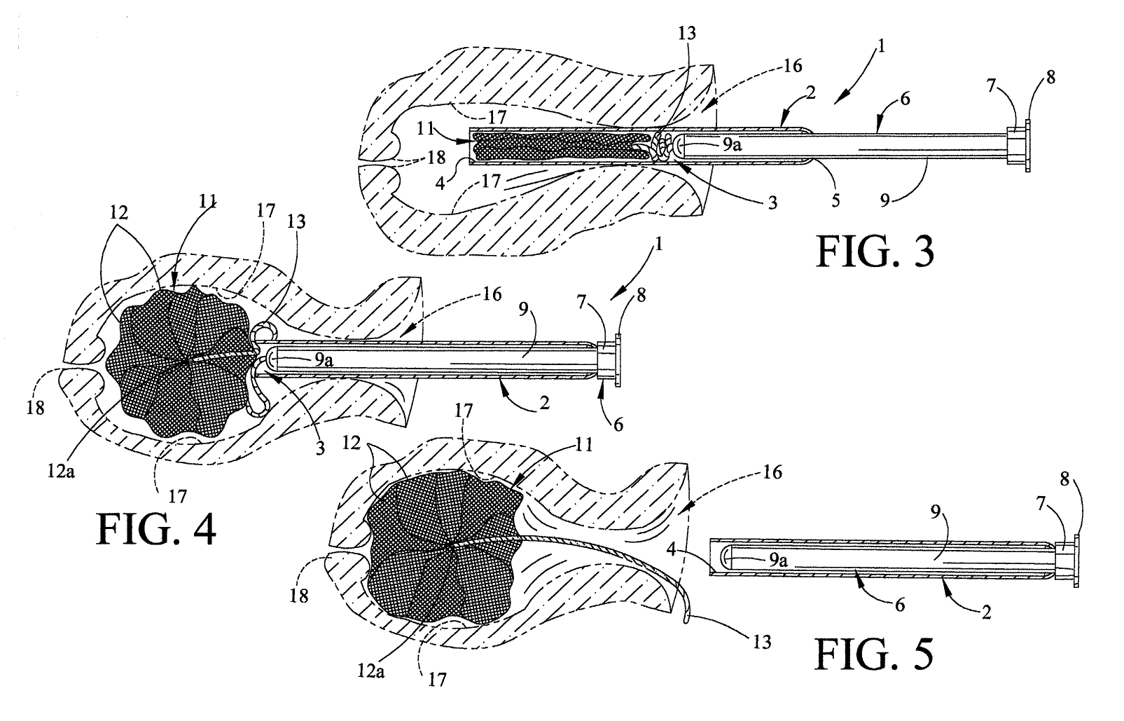

Referring initially to FIGS. 1-3 of the drawings, an illustrative embodiment of the device for collecting cellular and DNA specimens, hereinafter referred to as the device, of this invention is generally illustrated by reference numeral 1. The device 1 includes an elongated, hollow plunger housing 2, typically constructed of paper or plastic, in non-exclusive particular, and having a front opening 4 and a rear opening 5 which communicate with a housing interior 3 (FIG. 3). A plunger 6, typically having an elongated plunger shaft 9 and terminated on the rear end thereof by a plunger base 7 fitted with a plunger flange 8, is slidably disposed in the housing interior 3 of the plunger housing 2, with the typically rounded front end 9a of the plunger shaft 9 inserted in the rear opening 5 of the plunger housing 2, as illustrated in FIG. 2. A cell-collecting insert 11, shown in the functional, expanded configuration in FIG. 2, typically includes multiple, generally teardrop-shaped insert panels 12, the apices of which are sewn or otherwise attached to each other at a central attachment point 12a and extend outwardly therefrom in a compact, generally spherical array. The insert panels 12 can be constructed of any suitable flexible material having an expandable “memory”, including nylon and cellulose, in non-exclusive particular. A retrieval stem 13, such as nylon twine, in non-exclusive particular, may be attached to the insert 11 for purposes hereinafter described. As illustrated in FIG. 3, the insert 11 is normally confined in a compressed configuration in the housing interior 3 of the plunger housing 2, with at least a portion of the retrieval stem 13 typically extending through and captured in a central opening (not illustrated) provided in the front end 9a of the plunger shaft 9. In typical use of the device 1 as hereinafter described, the retrieval stem 13 may be useful for a female patient's retrieving the expanded insert 11, released from the plunger housing 2, from the vaginal opening 16 (FIG. 5) of her reproductive tract after use of the device 1.

Referring again to FIG. 3 and to FIGS. 4 and 5 of the drawings, in typical application of the device 1, a gynecological patient initially inserts the front opening 4 end of the elongated plunger housing 2 into the vaginal opening 16 of her reproductive tract, with the plunger 6 extending from the plunger housing 2 and the insert 11 disposed in the compressed configuration in the plunger housing 2, and the vaginal wall 17 partially grips the outside surface of the plunger housing 2, as illustrated in FIG. 3. The patient then discharges the cell-collecting insert 11, initially confined in the compressed configuration inside the housing interior 3, from the housing interior 3 through the front opening 4 of the plunger housing 2 by applying forward pressure against the plunger flange 8 until the plunger base 7 contacts the plunger housing 2 as illustrated in FIG. 4. Accordingly, as the front end 9a of the forwardly-advancing plunger shaft 9 pushes the compressed insert 11 from the housing interior 3 through the front opening 4 of the plunger housing 2, the flexible insert panels 12 expand outwardly to define the generally spherical insert 11 illustrated in FIG. 4, and the expanded insert 11 contacts substantially the entire diameter of both the circular upper vaginal wall 17 as well as the lower cervical wall 18 of the patient's reproductive tract. The patient then grasps and pulls the plunger housing 2 rearwardly to remove the plunger housing 2 and plunger 6, nested in the plunger housing 2, from the vaginal opening 16 as illustrated in FIG. 5 and advance the retrieval stem 13 from the central front opening (not illustrated) in the front end 9a of the plunger shaft 9. After the plunger housing 2 is completely removed from the vaginal opening 16, the extending end of the retrieval stem 13 typically releases from the tapered front end 9a of the plunger 6 and partially protrudes from the vaginal opening 16, as further illustrated in FIG. 5. As the expanded insert 11 remains in position against the upper vaginal wall 17 and the lower cervical wall 18 for a selected period of time, typically about eight hours or overnight, vaginal and cervical cells (not illustrated) as well as loose DNA from the upper vaginal wall 17 and the lower cervical wall 18 become detached from the vaginal wall 17 and cervical wall 18 and are trapped on the insert panels 12. At the end of the application period, the retrieval stem 13 can be grasped and pulled to retrieve the easily collapsible insert 11 from the vaginal opening 16, after which the insert 11 is typically placed in a suitable sample container (not illustrated) for transport to an appropriate medical facility. The vaginal and cervical cells, collected on the insert panels 12, are removed therefrom by means known to those skilled in the art and subjected to various cytologic studies and DNA analysis for the detection of various medical conditions such as cancer, sexually transmitted diseases and abnormal vaginal and cervical cytology.

It will be appreciated by those skilled in the art that the device for collecting cellular and DNA specimens of this invention is capable of contacting a large surface area of the vaginal and cervical wall to collect cells and DNA samples from these areas for cytological or chromosomal analysis. The various components of the device can be constructed in different sizes, and the expandible insert component of the device can be constructed in any size and geometrical shape which facilitates a large surface area of contact between the insert and the cellular surfaces.

While the preferred embodiments of the invention have been described above, it will be recognized and understood that various modifications can be made in the invention and the appended claims are intended to cover all such modifications which may fall within the spirit and scope of the invention.

Having described my invention with the particularity set forth above, what is claimed is:

1. A device for collecting cellular and DNA specimens from cellular surfaces, said device comprising:
a plunger housing for positioning adjacent to the cellular surfaces;
a plunger slidably disposed in said plunger housing;
an insert for positioning in a compressed configuration in said plunger housing, said insert releasably engaging said plunger housing when said insert is in said compressed configuration; and
wherein said insert expands spacially and contacts the cellular surfaces upon discharge of said insert from said plunger housing, responsive to sliding of said plunger in said plunger housing.

2. The device of claim 1 comprising a retrieval stem extending from said insert for retrieving said insert from the cellular surfaces.

3. The device of claim 1 wherein said insert is constructed of nylon.

4. The device of claim 3 comprising a retrieval stem extending from said insert for retrieving said insert from the cellular surfaces.

5. The device of claim 1 wherein said plunger comprises:
a plunger shaft having a front end inserted in said plunger housing and a rear end spaced from said front end;
a plunger base terminating said rear end of said plunger shaft; and
a plunger flange provided on said plunger base for pressing said plunger and sliding said plunger in said plunger housing.

6. The device of claim 5 comprising a retrieval stem extending from said insert for removably engaging said front and of said plunger shaft and retrieving said insert from the cellular surfaces.

7. The device of claim 5 wherein said insert is constructed of nylon.

8. The device of claim 7 comprising a retrieval stem extending from said insert for removably engaging said front and of said plunger shaft and retrieving said insert from the cellular surfaces.

9. A device for collecting cellular and DNA specimens from cellular surfaces, said device comprising:
a plunger housing for positioning adjacent to the cellular surfaces;
a plunger slidably disposed in said plunger housing;
an insert for positioning in a compressed configuration in said plunger housing, said insert comprising a central attachment point and a plurality of insert panels extending from said central attachment point;
wherein said insert releasably engages said plunger housing when said insert is in said compressed configuration; and
wherein said insert expands spatially and said insert panels assume a generally spherical configuration and contacts the cellular surfaces upon discharge of said insert from said plunger housing, responsive to sliding of said plunger in said plunger housing.

10. The device of claim 9 comprising a retrieval stem extending from said insert for retrieving said insert from the cellular surfaces.

11. The device of claim 9 wherein said insert panels of said insert are constructed of nylon.

12. The device of claim 11 comprising a retrieval stem extending from said insert for retrieving said insert from the cellular surfaces.

13. The device of claim 9 wherein said plunger comprises:
a plunger shaft having a front end inserted in said housing and a rear end spaced from said front end;
a plunger base terminating said rear end of said plunger shaft; and
a plunger flange provided on said plunger base.

14. The device of claim 13 comprising a retrieval stem extending from said insert for removably engaging said front and of said plunger shaft and retrieving said insert from the cellular surfaces.

15. The device of claim 13 wherein said insert panels of said insert are constructed of nylon.

16. The device of claim 15 comprising a retrieval stem extending from said insert for removably engaging said front and of said plunger shaft and retrieving said insert from the cellular surfaces.

17. A method of collecting cellular and DNA specimens from cellular surfaces, said method comprising:
providing a device comprising a plunger housing, a plunger slidably disposed in said plunger housing and an insert disposed in a compressed configuration in said plunger housing with said insert releasably engaging said plunger housing;
positioning said plunger housing adjacent to the cellular surfaces;
discharging said insert from said plunger housing by sliding said plunger in said plunger housing, wherein said insert expands and contacts the cellular surfaces;
maintaining said insert in contact with the cellular surfaces for at least about one hour; and
retrieving said insert from the cellular surfaces.

18. The method of claim 17 comprising providing a retrieval stem on said insert for said retrieving said insert from the cellular surfaces.

19. The method of claim 17 wherein said maintaining said insert in contact with the cellular surfaces for at least about one hour comprises maintaining said insert in contact with the cellular surfaces for about eight hours.

20. The method of claim 19 comprising providing a retrieval stem on said insert for said retrieving said insert from the cellular surfaces.

21. A method of collecting cellular and DNA specimens from cellular surfaces, said method comprising:
providing a device comprising a plunger housing, a plunger slidably disposed in said plunger housing and an insert disposed in a compressed configuration in said plunger housing with said insert releasably engaging said plunger housing, said insert comprising a central attachment point and a plurality of insert panels extending from said central attachment point;
positioning said plunger housing adjacent to the cellular surfaces;
discharging said insert from said plunger housing by sliding said plunger in said plunger housing, wherein said insert panels extend outwardly from said central attachment point to expand said insert and said insert assumes a substantially spherical configuration and contacts the cellular surfaces;
maintaining said insert in contact with the cellular surfaces for at least about one hour; and
retrieving said insert from the cellular surfaces.

22. The method of claim 21 comprising providing a retrieval stem on said insert for said retrieving said insert from the cellular surfaces.

23. The method of claim 21 wherein said maintaining said insert in contact with the cellular surfaces for at least about one hour comprises maintaining said insert in contact with the cellular surfaces for about eight hours.

24. The method of claim 23 comprising providing a retrieval stem on said insert for removably engaging said front and of said plunger shaft and retrieving said insert from the cellular surfaces.
